(19) United States
(12) Patent Application Publication
(10) Pub. No.: US 2025/0381416 A1
(43) Pub. Date: Dec. 18, 2025

(54) THERAPEUTIC DEVICE

(71) Applicant: Shenzhen Guangshu Medical Technology Co., Ltd, Shenzhen (CN)

(72) Inventors: ALAIN DIJKSTRA, Amstelveen (NL); KIM LAURENS MARKWAT, Den Haag (NL); MICHAEL KASBERGEN, Schoonhoven (NL)

(73) Assignee: Shenzhen Guangshu Medical Technology Co., Ltd, Shenzhen (CN)

(21) Appl. No.: 18/751,397

(22) Filed: Jun. 24, 2024

(30) Foreign Application Priority Data
Jun. 12, 2024 (CN) 2024213366481

(51) Int. Cl. A61N 5/067; A61N 5/06

(52) U.S. Cl. CPC A61N 5/067; A61N 2005/0626; A61N 2005/063; A61N 2005/0647; A61N 2005/0652; A61N 2005/066; A61N 2005/0661; A61N 2005/0663

(57) ABSTRACT

Embodiments of the present invention provide a therapeutic device. The therapeutic device includes an inner layer including a plurality of sensing locations and a plurality of stimulating locations constructed therewithin. Furthermore, the therapeutic device includes a plurality of stimulation elements located within the plurality of stimulating locations. The therapeutic device further includes a plurality of optical fibers including a plurality of respective first optical endpoints and a plurality of respective second optical endpoints, the plurality of first optical endpoints located within the plurality of sensing locations. Furthermore, the therapeutic device includes an optical sensor configured to receive electromagnetic radiation from the plurality of second optical endpoints. The therapeutic device also includes a control module configured to determine radiation characteristics of the electromagnetic radiation received by the optical sensor and activate one or more of the plurality of stimulation elements in correlation with the determined radiation characteristics.

### TECHNICAL FIELD

The present invention relates generally to therapeutic and recreational devices. More specifically, the present invention relates to devices that can deliver multiple kinds of stimulation such as electrical, heating, cooling, and irradiation, in correlation with determined skin parameter values using optical fibers for sensing applications.

### BACKGROUND ART

Therapeutic devices of several kinds for therapeutic and recreational purposes have been known in the art for quite some time. Such devices use electrodes, heating elements, cooling elements, and irradiation sources such as Light Emitting Diodes (LEDs) and lasers to deliver energy in one form or the other to a portion of the body for pain relief, muscle relaxation, skin rejuvenation, neural stimulation, etc. The therapeutic devices that are available in the art are generally controlled through feed-forward control. Therefore, it is generally hard to adapt the effect provided by such therapeutic devices in response to the changing physiological condition of a user during the treatment. There have been some solutions proposed in the art that deploy several kinds of sensors for determining localized values of parameters such as temperature, skin abnormalities, etc. for modulating the stimulation provided in specific regions of the skin of the user, thereby enabling a feedback control loop.

However, such solutions suffer from several drawbacks. For instance, the number and types of sensors that can be used are generally constrained by limitations posed by the weight, complexity, and cost of the resultant therapeutic device. In other words, if more than one parameter is needed to be determined, several different types of sensors may be installed for sensing temperature, pressure, visible abnormalities, etc. at several different locations in the therapeutic device. Moreover, the therapeutic device will likely be able to be adapted for a limited number of applications as different regions of the human body experience different types of maladies and therefore require different kinds of sensors with distinct properties, such as resolution, for sensing the same parameters, such as the temperature or surface abnormalities. Therefore, the construction of the therapeutic device that is adapted to be used as a waist belt will most likely be very different from the construction that can be used as an armband or a face mask.

Therefore, there is a need for a therapeutic device that overcomes the disadvantages and limitations associated with the prior art and provides a more satisfactory solution.

### OBJECTS OF THE INVENTION

Some of the objects of the invention are as follows:

An object of the present invention is to provide a therapeutic device that can be used for multiple purposes such as electrical stimulation, heating, cooling, vibratory massage, etc.

Another object of the present invention is to provide a therapeutic device that deploys localized sensing of skin parameter values such as skin temperature and skin abnormalities for enabling feedback-based closed-loop control of the therapeutic effects provided by the therapeutic device.

Another object of the present invention is to provide a therapeutic device that uses optical fibers as sensing elements to enable the closed-loop control of the therapeutic effects.

Another object of the invention is to provide a therapeutic device that uses radiation characteristics, such as intensity, wavelength, polarization, transit time, etc. of electromagnetic radiation transmitted through the optical fibers to determine the localized skin parameter values.

Another object of the invention is to provide a therapeutic device that combines additional auxiliary elements such as temperature-sensitive coatings and microcavity structures with the optical fibers to aid the determination of the localized skin parameter values.

Another object of the invention is to provide a therapeutic device that can be embodied in several different forms such as face masks, armbands, waist belts, etc. with minimal structural redesign and operational reconfiguration.

Another object of the invention is to provide a therapeutic device that deploys Artificial Intelligence (AI) and Machine Learning (ML) algorithms trained on historical data and remodeled using real-time data to generate relatively more precise and effective therapeutic effects that are customized to suit specific physiological characteristics of a user receiving the therapy.

### SUMMARY OF THE INVENTION

According to a first aspect of the present invention, there is provided a therapeutic device. The therapeutic device includes an inner layer including a plurality of sensing locations and a plurality of stimulating locations constructed therewithin, the inner layer configured to be in contact with the skin of a user. Furthermore, the therapeutic device includes a plurality of stimulation elements located within the plurality of stimulating locations. The therapeutic device further includes a plurality of optical fibers including a plurality of respective first optical endpoints and a plurality of respective second optical endpoints, the plurality of first optical endpoints located within the plurality of sensing locations. Furthermore, the therapeutic device includes an optical sensor configured to receive electromagnetic radiation traveling from the plurality of first optical endpoints to the plurality of second optical endpoints. Also, the therapeutic device includes a control module configured to determine radiation characteristics of the electromagnetic radiation received by the optical sensor and activate one or more of the plurality of stimulation elements in correlation with the determined radiation characteristics.

In one embodiment of the invention, the therapeutic device further includes a light source configured to provide incident radiation to the plurality of optical fibers through the plurality of second optical endpoints, wherein the electromagnetic radiation received by the optical sensor is reflected radiation from the plurality of first optical endpoints.

In one embodiment of the invention, one or more of the plurality of sensing locations at least partially overlap with one or more of the plurality of stimulating locations.

In one embodiment of the invention, the therapeutic device is embodied as one or more of a face mask, a waistbelt, and an armband.

In one embodiment of the invention, the therapeutic device is made from one or more flexible materials.

In one embodiment of the invention, the plurality of first optical endpoints are distributed amongst the plurality of sensing locations, and the plurality of second optical endpoints are bundled in the form of an optical cord.

In one embodiment of the invention, one or more of the plurality of optical fibers includes microcavity structures for temperature sensing.

In one embodiment of the invention, the radiation characteristics are selected from a group consisting of intensity, phase, polarization, wavelength, transit time, and combinations thereof.

In one embodiment of the invention, the plurality of stimulation elements is selected from a group consisting of irradiation sources, heating elements, cooling elements, vibration elements, ultrasonic wave generators, electrodes, and combinations thereof.

In one embodiment of the invention, the irradiation sources are selected from a group consisting of Light Emitting Diodes (LEDs), and lasers.

In one embodiment of the invention, the irradiation sources are configured to emit electromagnetic radiation in a range of wavelengths varying between 300 nm and 1200 nm.

In one embodiment of the invention, one or more of the plurality of first optical endpoints include a parameter-sensitive coating thereupon, the parameter-sensitive coating being sensitive to one or more physiological parameters of the user.

In one embodiment of the invention, the parameter-sensitive coating includes a thermographic phosphor.

In one embodiment of the invention, the control module is further configured to access a location database, the location database including a plurality of first predefined location coordinate sets of the plurality of respective sensing locations and a plurality of second predefined location coordinate sets of the plurality of respective stimulating locations, about a reference coordinates system.

In one embodiment of the invention, the control module is further configured to determine at least one physiological parameter at each one of the plurality of first location coordinate sets indicative of the plurality of respective sensing locations, from the determined radiation characteristics, and store the determined at least one physiological parameter of each one of the plurality of first location coordinate sets, in a storage device.

In one embodiment of the invention, the at least one physiological parameter is selected from a group consisting of temperature, pressure, strain, and skin abnormalities.

In one embodiment of the invention, the control module is configured to determine the at least one physiological parameter by utilizing interferometers, Fiber Bragg gratings, confocal microscopy, Optical Coherence Tomography (OCT), fluorescence spectroscopy, Brillouin scattering, and Raman scattering.

In one embodiment of the invention, the control module is configured to utilize Machine Learning (ML) algorithms in combination with historical reference data for Artificial Intelligence (AI) based determination of the at least one physiological parameter.

According to a second aspect of the present invention, there is provided a therapeutic device. The therapeutic device includes an inner layer including a plurality of sensing locations and a plurality of stimulating locations constructed therewithin, the inner layer configured to be in contact with the skin of a user. Furthermore, the therapeutic device includes a plurality of stimulation elements located within the plurality of stimulating locations. The therapeutic device further includes a plurality of optical fibers including a plurality of respective first optical endpoints and a plurality of respective second optical endpoints, the plurality of first optical endpoints located within the plurality of sensing locations. The therapeutic device further includes an optical sensor configured to receive electromagnetic radiation traveling from the plurality of first optical endpoints to the plurality of second optical endpoints. Furthermore, the therapeutic device includes a light source configured to provide incident radiation to the plurality of optical fibers through the plurality of second optical endpoints, wherein the electromagnetic radiation received by the optical sensor is reflected radiation from the plurality of first optical endpoints. The therapeutic device also includes a control module configured to determine radiation characteristics of the electromagnetic radiation received by the optical sensor and activate one or more of the plurality of stimulation elements in correlation with the determined radiation characteristics.

According to a third aspect of the present invention, there is provided a method of providing therapy. The method includes providing a therapeutic device. The therapeutic device includes an inner layer including a plurality of sensing locations and a plurality of stimulating locations constructed therewithin, the inner layer configured to be in contact with the skin of a user. The therapeutic device further includes a plurality of stimulation elements located within the plurality of stimulating locations. Furthermore, the therapeutic device includes a plurality of optical fibers including a plurality of respective first optical endpoints and a plurality of respective second optical endpoints, the plurality of first optical endpoints located within the plurality of sensing locations. The therapeutic device further includes an optical sensor configured to receive electromagnetic radiation traveling from the plurality of first optical endpoints to the plurality of second optical endpoints. Also, the therapeutic device includes a control module configured to determine radiation characteristics of the electromagnetic radiation received by the optical sensor and activate one or more of the plurality of stimulation elements in correlation with the determined radiation characteristics. The method further includes receiving, by the optical sensor, the electromagnetic radiation traveling from the plurality of first optical endpoints to the plurality of second optical endpoints. Furthermore, the method includes determining, by the control module, the radiation characteristics of the electromagnetic radiation received by the optical sensor. The method also includes activating, by the control module, the one or more of the plurality of stimulation elements in correlation with the determined radiation characteristics.

In the context of the specification, the term “interferometer” refers to fiber optic interferometers that divide an incident beam into a reference beam and a sensing beam. The sensing beam is affected by a parameter of interest and then is made to interfere with the reference beam. The interferometer is configured to analyze an interference pattern generated due to interference of the affected sensing beam and the reference beam to determine the parameter of interest. Commonly used fiber optic interferometers include Michelson interferometer, Fabry-Perot interferometer, Sagnac interferometer, Mach- Zehnder interferometer, Modal interferometer, Moiré interferometer, and White light interferometer.

In the context of the specification, the phrase “Fiber Bragg gratings (FBGs)” refers to small segments of optical fibers (order of a few millimeters long) with permanent modifications to their refractive indices in a manner that they act as wavelength-specific mirrors, thereby reflecting specific wavelengths while transmitting others. FBGs are used for sensing applications because changes in the environment of an optical fiber, such as changes in temperature, pressure, and strain can cause shifts in reflected wavelengths. The amount of shift from a known wavelength may then be correlated with the temperature, pressure, and strain or changes therein, in the environment of the optical fiber.

In the context of the specification, the phrase “Optical Coherence Tomography (OCT)” refers to a technique that combines optical fibers, interferometers, and computing hardware to generate cross-sectional images of tissues under examination. OCT is similar to interferometry with an additional step of utilizing a computer-based algorithm to analyze the resultant interference pattern and generate cross-sectional images of the tissues under examination. The generated cross-sectional images may then be displayed on a display device.

In the context of the specification, the phrase “fluorescence spectroscopy” refers to a technique that involves exciting known substances with incident light to enable them to exhibit fluorescence (wherein the light of a different wavelength when compared with incident light is emitted by the known substance) and receiving the emitted light to analyze the properties of the known substance. Fluorescence spectroscopy is used in sensing applications in conjunction with optical fibers (used as mediums for transmitting the incident light and receiving the emitted light) as changes in the environment of the known substance, such as changes in the temperature, pH, etc. may further shift the wavelength of the light emitted by the known substance. The shifts in the wavelength when compared with wavelengths associated with the reference environment of the known substance may then be calibrated as a measure of the changes in the environment.

In the context of the specification, the phrase “Brillouin scattering or Brillouin light scattering (BLS)” refers to a phenomenon resulting from the interaction of light with sound waves causing the light to scatter in forward and backward directions. The backward scattered light experiences a frequency shift referred to as the Brillouin shift. The Brillouin shift is indicative of the characteristics of the sound waves and the physical and chemical properties of the material itself. The scattered light is analyzed using a customized spectrometer to determine the Brillouin spectrum. The BLS may be combined with optical fibers for sensing applications as changes in the environment of the optical fiber, such as changes in the temperature, pressure, etc. would cause changes in the Brillouin shift of the material of the optical fiber when compared with Brillouin shift in reference environmental conditions.

In the context of the specification, the phrase “Raman scattering or Raman effect” refers to the inelastic scattering of light caused by the interaction and exchange of energy between photons of light and a given molecule. The scattered light may then be analyzed to generate a Raman spectrum indicative of the chemical and physical properties of the molecule itself. Raman effect may be combined with optical fibers for sensing applications as changes in the environment of the optical fiber, such as changes in the temperature, pressure, etc. would cause changes in the Raman spectrum of the material of the optical fiber when compared with the Raman spectrum in reference environmental conditions.

In the context of the specification, the term “processor” refers to one or more of a microprocessor, a microcontroller, a general-purpose processor, a Field Programmable Gate Array (FPGA), a Neural Processing Unit (NPU), a Graphics Processing Unit (GPU), a Tensor Processing Unit (TPU), an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of the specification, the term “historical” in the execution of a command refers to anything about a time instant(s) that is earlier than a time instant of an initiation of the command.

In the context of the specification, the term, “real-time”, refers to without intentional delay, given the processing limitations of hardware/software/firmware involved and the time required to accurately measure/receive/process/transmit data as practically possible.

In the context of this specification, terms like “light”, “radiation”, “irradiation”, “emission” and “illumination”, etc. refer to electromagnetic radiation in frequency ranges varying between the Ultraviolet (UV) frequencies and Infrared (IR) frequencies and wavelengths (including all visible light frequencies and wavelengths), wherein the range is inclusive of UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-a (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, the term “polymer” refers to a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, the phrase “diaphanous material” refers to a material that allows at least a portion of one or more forms of electromagnetic radiation (such as Infrared, Ultraviolet, X-rays, Visible Light, Microwaves, Radio Waves, etc.) to pass through them. The diaphanous materials can be transparent (allowing one or more forms of electromagnetic radiation to pass through with minimal scattering) or translucent (allowing one or more forms of electromagnetic radiation to pass through with appreciable diffusion or scattering). Diaphanous materials can be dense, like glass, or have an open structure, like wire mesh or a woven fabric.

In the context of the specification, “Light Emitting Diodes (LEDs)” refer to semiconductor diodes capable of emitting electromagnetic radiation when supplied with an electric current. LEDs are characterized by their superior power efficiencies, smaller sizes, rapidity in switching, physical robustness, and longevity when compared with incandescent or fluorescent lamps. In that regard, one or more LEDs may be through-hole type LEDs (generally used to produce electromagnetic radiations of red, green, yellow, blue, and white colors), Surface Mount Technology (SMT) LEDs, Bi-color LEDs, Pulse Width Modulated RGB (Red-Green-Blue) LEDs, and high-power LEDs, etc.

Materials used in the LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium-doped potassium fluorosilicate in combination with a blue LED to generate red light. Additionally, near Ultraviolet (UV) LEDs may be combined with europium-based phosphors to generate red and blue lights and copper and zinc-doped zinc sulfide-based phosphors to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a front view of a therapeutic device, in accordance with an embodiment of the present invention;

FIG. 1B illustrates an exploded view of a wearable pad portion of the therapeutic device of FIG. 1A;

FIG. 2A illustrates the therapeutic device of FIG. 1A in communication with a user computing device, in accordance with an embodiment of the present invention;

FIG. 2B illustrates a logical diagram of a control architecture of the therapeutic device, in accordance with an embodiment of the present invention;

FIG. 3A illustrates a front view of a therapeutic device, in accordance with another embodiment of the present invention;

FIG. 3B illustrates a front view of a therapeutic device, in accordance with another embodiment of the present invention;

FIG. 3C illustrates an exploded view of the therapeutic device of FIG. 3B, in accordance with an embodiment of the present invention;

FIG. 4A illustrates a plurality of optical fibers sensing at least one parameter indicative of a skin condition in a facial region of a user, in accordance with an embodiment of the present invention;

FIG. 4B illustrates the plurality of optical fibers sensing the at least one parameter indicative of the skin condition in an arm region of a user, in accordance with an embodiment of the present invention;

FIG. 5 illustrates a method of providing therapy using a therapeutic device, in accordance with an embodiment of the present invention; and

FIG. 6A illustrates an optical sensor receiving electromagnetic radiation traveling through the plurality of optical fibers, in accordance with an embodiment of the present invention;

FIG. 6B illustrates a segment of a matrix of radiation characteristics derived from the electromagnetic radiation received by the optical sensor, in accordance with an embodiment of the present invention;

FIG. 7 illustrates a user wearing several different embodiments of the therapeutic device depicting several possible application areas of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention provide a therapeutic device (hereinafter also referred to as “the device”). The device is envisaged to be a wearable device such as a face mask, an armband, a waistbelt, etc. Furthermore, the device is envisaged to be enabled with feedback control using optical fibers located in sensing locations for sensing several parameters such as temperature, pressure, strain, visual skin abnormalities, etc. The therapy may be provided using several stimulation elements located in stimulating locations. The stimulation elements may include irradiation sources such as LEDs or lasers, heating elements, cooling elements, vibration elements, ultrasonic wave generators, electrodes, and combinations thereof. In that regard, the optical fibers may form the innermost portion of the therapeutic device with ends either in direct contact with the skin of a user or in closest relative proximity. Furthermore, parameter-sensitive coatings, such as phosphor coatings for temperature measurement, may be provided at the ends of the optical fibers, that are close to the skin, to amplify information sensed by the optical fibers.

Electromagnetic radiation traveling from the ends that are proximal to the skin of the user to ends that are distal to the skin of the user may be received by an optical sensor. In several embodiments, the therapeutic device may further include a light source with known characteristics to form a reference for the measurement of variations in the known characteristics to deduce the intended parameter values. A control module would then determine the radiation characteristics of the electromagnetic radiation received by the optical sensor. The radiation characteristics may include intensity, phase, polarization, wavelength, frequency, transit time, and combinations thereof. In several alternate scenarios, especially where the light source is being used, the radiation characteristics determined by the control module may include variations or changes in the intensity, phase, polarization, wavelength, frequency, transit time, and combinations thereof, from respective known values.

The control module may then determine values of one or more of the physiological parameters such as the temperature, pressure, strain, and skin abnormalities from the determined radiation characteristics. In that regard, the control module may be programmed to deploy several techniques such as utilizing interferometers, Fiber Bragg gratings, confocal microscopy, Optical Coherence Tomography (OCT), fluorescence spectroscopy, Brillouin scattering, and Raman scattering, to determine the values of the one or more physiological parameters. In addition, the control module may be enabled with Machine Learning (ML) algorithms and pre-trained on large historical datasets to deploy Artificial Intelligence (AI) techniques to determine the values of the physiological parameters. The control module would then activate one or more stimulation elements in correlation with the determined radiation characteristics and/or the determined physiological parameters. To enable the control module to establish correlations between the determined radiation characteristics and the stimulation elements, the stimulating locations and the sensing locations may be defined about a reference coordinate system and location coordinate sets indicative of the stimulating locations and the sensing locations may stored in a location database that may be accessible to the control module.

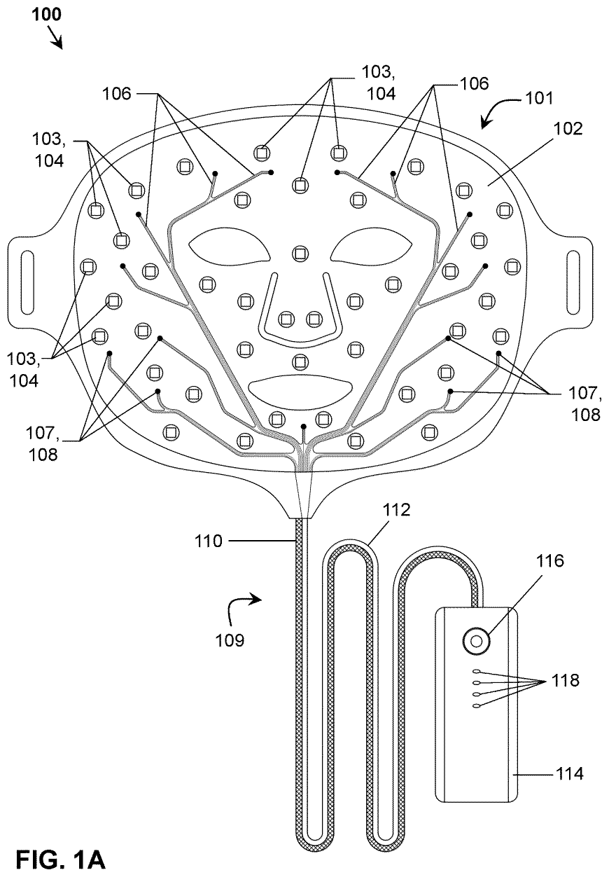

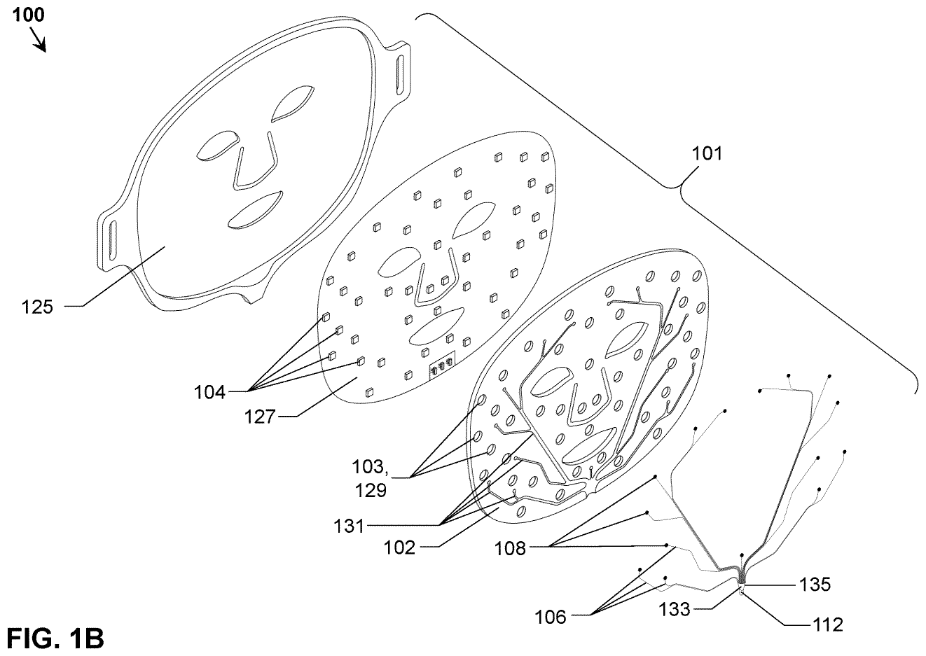

Several embodiments of the present invention will now be discussed in detail taking FIGS. 1-7 as reference.

FIG. 1A illustrates a front view of a therapeutic device 100 (hereinafter also referred to as “the device 100”), in accordance with an embodiment of the present invention. The device 100 has been embodied as a face mask. However, several alternate constructions of the device 100, such as armbands and waist belts, are also within the scope of the invention, as will be presented later in the discussion. The device 100 includes a wearable pad 101 in the shape of a face mask. The wearable pad 101 includes an inner layer 102, an outer layer, and a flexible Printed Circuit Board (PCB) located between the inner layer 102 and the outer layer (See FIG. 1B). The inner layer 102 is configured to be in contact with the skin of a user during usage of the device 100. Furthermore, inner layer 102 includes a plurality of stimulating locations 103 from where therapeutic stimulation is envisaged to be provided to the user. In that regard, the plurality of stimulating locations 103 are envisaged to include a plurality of respective stimulation elements 104.

The plurality of stimulation elements 104 may be selected from a group consisting of irradiation sources, heating elements, cooling elements, vibration elements, ultrasonic wave generators, electrodes, and combinations thereof. Furthermore, irradiation sources may be selected from a group consisting of Light Emitting Diodes (LEDs), and lasers. Additionally, the irradiation sources may be configured to operate in one or more of a pulse mode and a continuous mode. In the case of a stimulation element being an irradiation source, the stimulation element would be emitting electromagnetic radiation. In the case of a stimulation element being a vibration element, the stimulation element would be a vibrating head connected to an eccentric mass rotating motor or a linear resonant motor.

In case of a stimulation element being a heating element, the stimulation element may be selected from a group consisting of metal heating elements, ceramic heating elements, semiconductor heating elements, thick film heating elements, polymer-based heating elements, composite heating elements, and combination heating elements. In the case of a stimulation element being a cooling element, the stimulation element may be a thermoelectric cooler, also known as a Peltier heat pump. In the case of a stimulation element being an ultrasonic wave generator, a wave generation head may consist of a quartz crystal fused with a metal plate. The quartz crystal may produce ultrasonic waves due to the piezoelectric effect. Ultrasonic wave therapy may be used in applications such as treatment of chronic pain, improvement in blood circulation, and tissue repair. In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the body of a user.

TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

Furthermore, the inner layer 102 includes a plurality of sensing locations 107. The plurality of sensing locations 107 includes a plurality of respective first optical endpoints 108 of a plurality of respective optical fibers 106 of the device 100. In several embodiments of the invention, although not bindingly, one or more of the plurality of stimulating locations 103 may at least partially overlap with one or more of the plurality of sensing locations 107. Furthermore, FIG. 1A illustrates a data cable 110 that communicably connects the wearable pad 101 with a remote controller 114. The data cable 110 in that regard may be selected from multi-core conductors, optical fibers, co-axial cables, twisted pair cables, and the like. Also, the plurality of optical fibers 106 emanates from an optical cord 112, in which at least portions of the plurality of optical fibers 106 are bundled together. The data cable 110 and the optical cord 112 may be coupled together to generate a data transfer cord 109 that connects the remote controller 114 with the wearable pad 101.

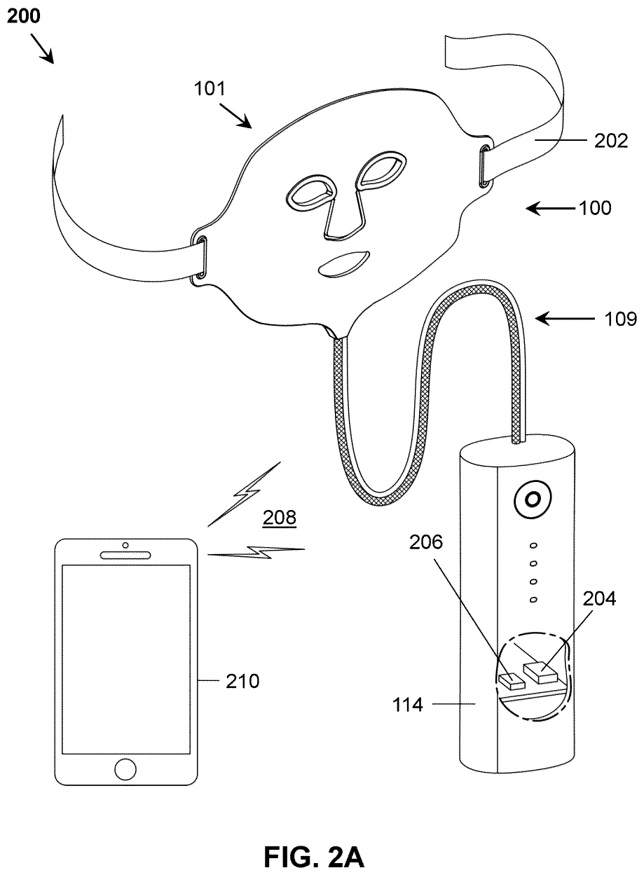

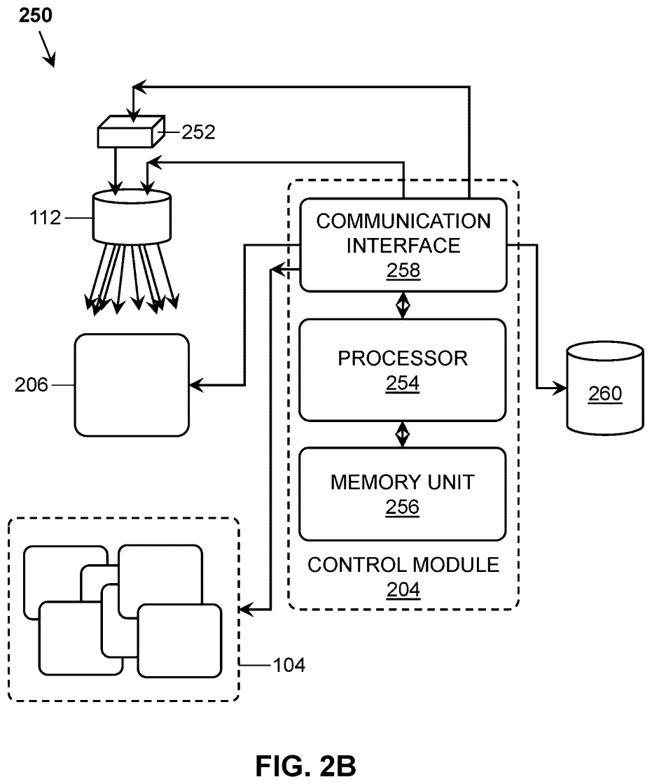

The remote controller 114 further includes a user interface 116 that may be used for several purposes such as switching the device 100 between on and off conditions, selecting between different predefined modes of operation, increasing or decreasing the intensity of the therapy being provided, broadcasting Media Access Control (MAC) address for connecting with a user computing device (See FIG. 2), and the like. The user interface 116 in that regard may be embodied as a touch screen (resistive or capacitive type), a single control button, or a group of control toggles. The remote controller 114 also includes several indicator units 118 that may be embodied as display screens, LEDs, and/or speaker units, and provide audiovisual information to the user about several operating parameters of the device 100. The operating parameters may be selected from a group consisting of the current state of the device 100 (on, off, or standby), mode of operation (continuous or pulsating), type of therapy being provided (heating, cooling, irradiation, vibratory massage, or combinations thereof), time of therapy (time elapsed, or time remaining), and state of charge of batteries powering the device 100.

FIG. 1B illustrates an exploded view of the wearable pad 101. As discussed before, the wearable pad 101 includes the inner layer 102, the outer layer 125, and a flexible PCB 127. It is to be noted that the inner layer 102, the outer layer 125, and the flexible PCB 127 are envisaged to be made up of flexible materials. For example, the inner layer 102 may be made up of skin-friendly flexible diaphanous materials such as hydrogels, polyurethanes, silicones, polyethylene naphthalate, etc. The outer layer 125 may be opaque to prevent radiation leakage and may be made up of knitted fabrics, silks, polydimethylsiloxane (PDMS), hydrogels, polyurethanes, silicones, polyethylene naphthalate, etc. Whereas the flexible PCB 127 may be made up of conductor materials, such as copper, aluminum, and nickel, deposited upon polymers such as polyimide (PI), polyester (PET), polyethylene naphthalate, etc. Epoxy and pressure-sensitive adhesives may be used to bind the layers, and an overlay may be provided over the deposited conductor layer to prevent the conductor layer from mechanical and/or chemical damage and/or abrasion.

Furthermore, the inner layer 102 includes a plurality of apertures 129 including holes and/or slots at the plurality of respective stimulating locations 103. The plurality of apertures 129 is configured to accommodate the plurality of respective stimulation elements 104. In several embodiments of the invention, the plurality of stimulation elements 104 may be surface mounted onto the flexible PCB 127. The inner layer 102 also includes a plurality of fiber channels 131 configured to accommodate the plurality of respective optical fibers 106. The plurality of optical fibers 106 includes the plurality of respective first optical endpoints 108 and a plurality of respective second optical endpoints 133 located inside a cone structure 135 of the optical cord 112. In several embodiments of the invention, the plurality of second optical endpoints 133 may be located within the remote controller 114 just above an optical sensor (See FIG. 2). The plurality of optical fibers 106 are envisaged to act as remote sensors with electromagnetic radiation traveling within the plurality of optical fibers 106 envisaged to be carrying information concerning at least one physiological parameter (such as temperature, pressure, strain, and other visual skin abnormalities) associated with the skin of the user.

Radiation characteristics of the electromagnetic radiations traveling within the plurality of optical fibers 106 may provide information about the at least one physiological parameter. For example, the intensity of the electromagnetic radiation may be affected by changes in the temperature, pressure, or strain. Similar to the intensity, the phase of electromagnetic radiation may be sensitive to environmental changes like vibrations or acoustic waves. Inherent polarization of electromagnetic radiation may be affected by external factors such as magnetic fields or electric currents. Some fiber optic materials exhibit color shifts or changes in wavelengths when exposed to specific chemicals or temperature variations. Moreover, the transit time of the electromagnetic radiation within the plurality of optical fibers 106 is also affected by changes in strain, pressure, etc.

In that regard, the plurality of optical fibers 106 may be further adapted in several ways to amplify the effects that the physiological parameters may have on the plurality of optical fibers. For example, doping the fiber optic material with certain known dopants would allow the plurality of optical fibers 106 to identify specific chemical molecules and temperature variations. Etching or removing a portion of the cladding material of the plurality of optical fibers 106 would enhance their sensitivity to changes in pressure and strain. Furthermore, microcavity structures, i.e., small air pockets may be introduced in the plurality of optical fibers 106 that may be configured to trap light creating resonant cavities. The resonant cavities are sensitive to temperature changes. Moreover, the plurality of first optical endpoints 108 may be provided with parameter-sensitive coatings, such as a thermographic phosphor coating, that are sensitive to one or more physiological parameters of the user.

FIG. 2A illustrates the therapeutic device 200 of FIG. 1A in communication with a user computing device 210, in accordance with an embodiment of the present invention. The device 100 is also depicted to include a fastening arrangement 202 configured to fasten the wearable pad 101 to a body portion (such as the face, the waist, or the arm) of the user. The user computing device 210 may be selected from a group consisting of smartphones, tablet PCs, notebook PCs, and the like. Moreover, the user computing device 210 is configured to communicate with a control module 204 of the remote controller 114 through a communication network 208. The remote controller 114 also includes an optical sensor 206 configured to receive the electromagnetic radiation traveling from the plurality of first optical endpoints 108 to the plurality of respective second optical endpoints 133. The optical sensor 206 may be selected from a group consisting of intensity-based sensors, phased-based sensors, wavelength-based sensors, polarization-based sensors, evanescent wave sensors, and combinations thereof.

FIG. 2B illustrates a logical diagram of a control architecture 250 of the therapeutic device 100, in accordance with an embodiment of the present invention. The control module 204 includes a processor 254, a memory unit 256 configured to store machine-readable instructions, and a communication interface 258. Furthermore, the control module 204 is coupled to a storage device 260 through the communication interface 258. The storage device 260 is configured to store static and dynamic data received or generated during the working of the device 100. In addition, in several embodiments, the device 100 may also include a light source 252 configured to provide incident light for sensing applications deploying techniques involving the use of interferometers, Optical Coherence Tomography (OCT), Raman scattering, fluorescence spectroscopy, Fiber Bragg gratings, confocal microscopy, etc. as will be discussed in the following discussion. In the embodiments involving the use of the light source 252, the light source 252 is configured to provide incident radiation to the plurality of optical fibers 106 through the plurality of respective second optical endpoints 133. Furthermore, the electromagnetic radiation received by the optical sensor 206 is reflected radiation from the plurality of first optical endpoints 108.

The light source 252 may be selected from several different available constructions. For example, LEDs may be used in the light source 252 owing to LEDs being capable of generating a wide range of wavelengths and having higher durability and lower manufacturing and operating costs. Another possible choice for the light source 252 may be super luminescent diodes (SLDs) with relatively broader spectral bandwidth and higher output power when compared with LEDs. SLDs are very pertinent to Raman scattering and Brillouin scattering-based sensors. Lasers used as the light source 252 provide incident beams with very high coherence for interferometric sensors and fiber Bragg grating sensors. Halogen lamps or xenon lamps may be used for fluorescence-based sensors. Ultraviolet (UV) light sources such as excimer lasers or mercury lamps may be used as the light source 252 for applications involving biomolecule detection.

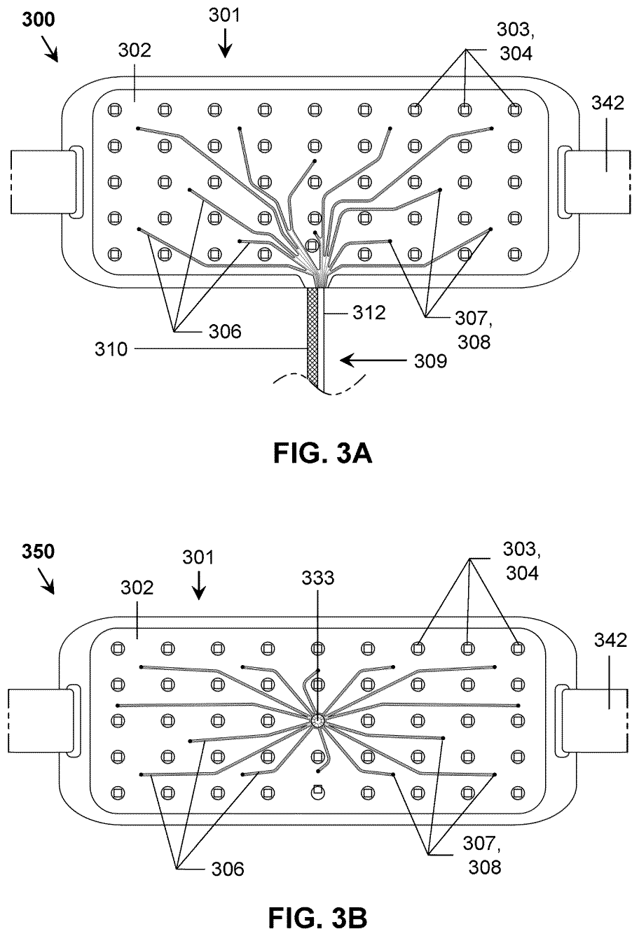

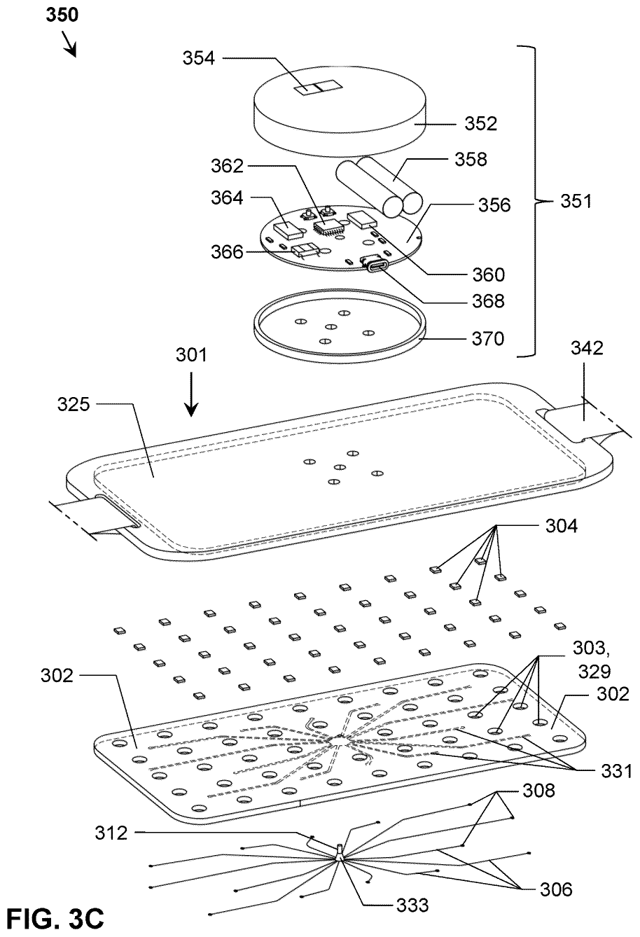

FIG. 3A illustrates a front view of a therapeutic device 300 (hereinafter also referred to as “the device 300”), in accordance with another embodiment of the present invention. The device 300 has been embodied as an armband or a waistbelt including a fastening arrangement 342. In equivalence to the device 100, the device 300 includes a wearable pad 301, an inner layer 302, a plurality of stimulation elements 304 located in a plurality of respective sensing locations 303 constructed within the inner layer 302, a plurality of optical fibers 306 with a plurality of respective first optical endpoints 308 located in a plurality of respective sensing locations 307 constructed within the inner layer 302. A data transfer cord 309 includes an optical cord 312 and a data cable 310. The data transfer cord 309 connects the wearable pad 301 with a corresponding remote controller (not shown). The device 300 may be controlled using the same remote controller 114 of the device 100 or another remote controller of equivalent construction.

FIG. 3B illustrates a front view of a therapeutic device 350 (hereinafter also referred to as “the device 350”), in accordance with another embodiment of the present invention. The device 350 is identical in many ways to the device 300, with the exception that the device 350 does not include a remote controller. On the contrary, a standalone controller is configured to be directly coupled to the wearable pad 301 (See FIG. 3C) and receive the electromagnetic radiation traveling between the plurality of first optical endpoints 308 and a plurality of respective second optical endpoints 333 of the plurality of respective optical fibers 306. The plurality of first optical endpoints 308 is distributed amongst the plurality of sensing locations 307.

FIG. 3C illustrates an exploded view of the therapeutic device 350 of FIG. 3B, in accordance with an embodiment of the present invention. The device 350 includes a standalone controller 351 configured to be electrically coupled to the wearable pad 301. The standalone controller 351 includes an upper housing portion 352 configured to couple with a lower housing portion 370 of the standalone controller 351. Furthermore, the standalone controller 351 includes a user interface 354, a power unit 358 including rechargeable or replaceable batteries, a PCB 356, a processor 362, a memory unit 366, an optical sensor 360, a light source 364 that is equivalent to the light source 252, and a communication interface 368 which may also double as charging port for wired charging of rechargeable batteries in the power unit 358. The processor 362, the memory unit 366, and the communication interface 368 together constitute a control module for the device 350. Furthermore, FIG. 3C illustrates an outer layer 325 of the wearable pad 301, a plurality of apertures 329 in the inner layer 302 including holes and/or slots at the plurality of respective stimulating locations 303. Furthermore, the inner layer 302 includes a plurality of fiber channels 331 for accommodating the plurality of respective optical fibers 306. Also, the plurality of second optical endpoints 333 is bundled in the form of the optical cord 312.

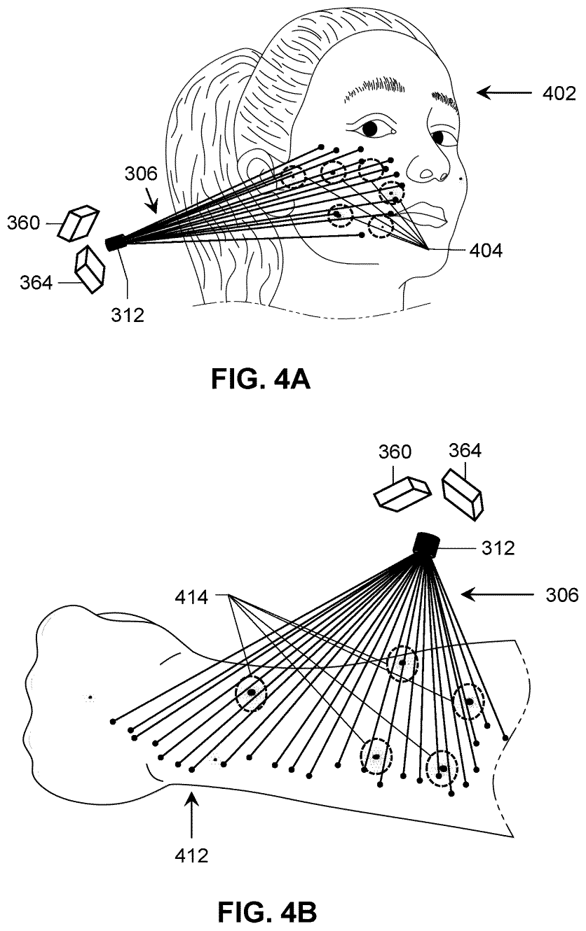

FIG. 4A illustrates the plurality of optical fibers 306 sensing at least one parameter indicative of a skin condition in a facial region 402 of a user, in accordance with an embodiment of the present invention. The optical cord 312 is configured to be illuminated by the light source 364 and the optical sensor 360 is configured to receive the reflected radiation from the facial region 402 of the user. The facial region 402 has several lesions and abnormalities 404 such as bumps, cracks, redness, freckles, etc. Such lesions and abnormalities would induce increased strain and pressure locally. Moreover, redness will have a deeper color signature than the region surrounding it. In certain lesions, the local temperature may be slightly higher due to inflammation. Therefore, light reflected from the lesions and abnormalities will experience changes in intensity, polarization, phase, transit time, etc. when compared with a reference beam. The aforementioned changes may then be captured by the optical sensor 360.

FIG. 4B illustrates the plurality of optical fibers 306 sensing the at least one parameter indicative of the skin condition in an arm 412 region of a user, in accordance with an embodiment of the present invention. The arm region 412 includes several lesions and abnormalities 414. The lesions and abnormalities 414 may exhibit similar effects on an incident sensing beam as exhibited by the lesions and abnormalities 404. In several alternate embodiments, the light source 364 may not be required, and illumination provided by ambient conditions may be enough to generate a reference beam and a sensing beam.

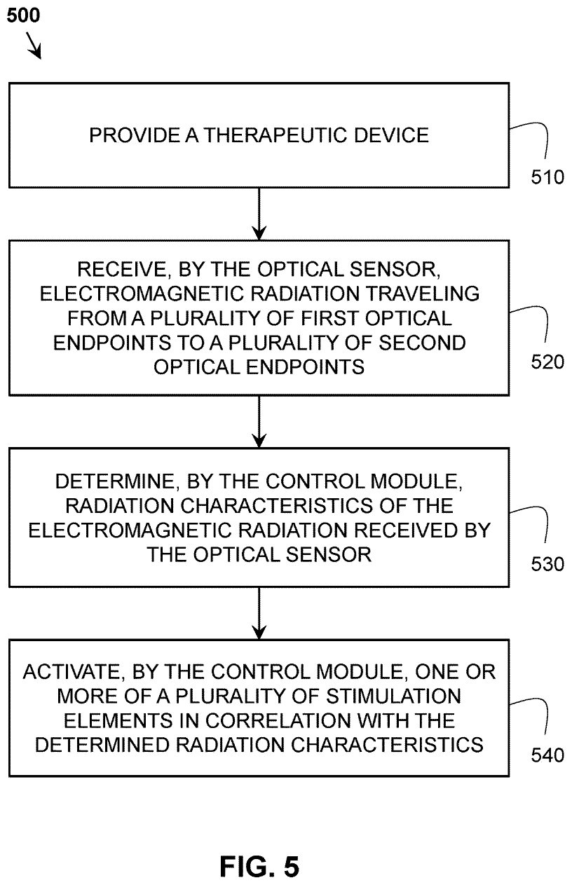

FIG. 5 illustrates a method 500 of providing therapy using the therapeutic device 100, 300, or 350, in accordance with an embodiment of the present invention. The method 500 begins at Step 510 when the therapeutic device 100, 300, or 350 is provided. At Step 520, the optical sensor 206 or 360 receives the electromagnetic radiation traveling from the plurality of first optical endpoints 108 or 308 to the plurality of second optical endpoints 133 or 333, respectively. In several embodiments of the invention, the electromagnetic radiation received by the optical sensor 206 or 360 may be a reflected sensing beam. The sensing beam would have been generated by splitting an incident beam generated by the light source 252 or 364, respectively, into a reference beam and a sensing beam.

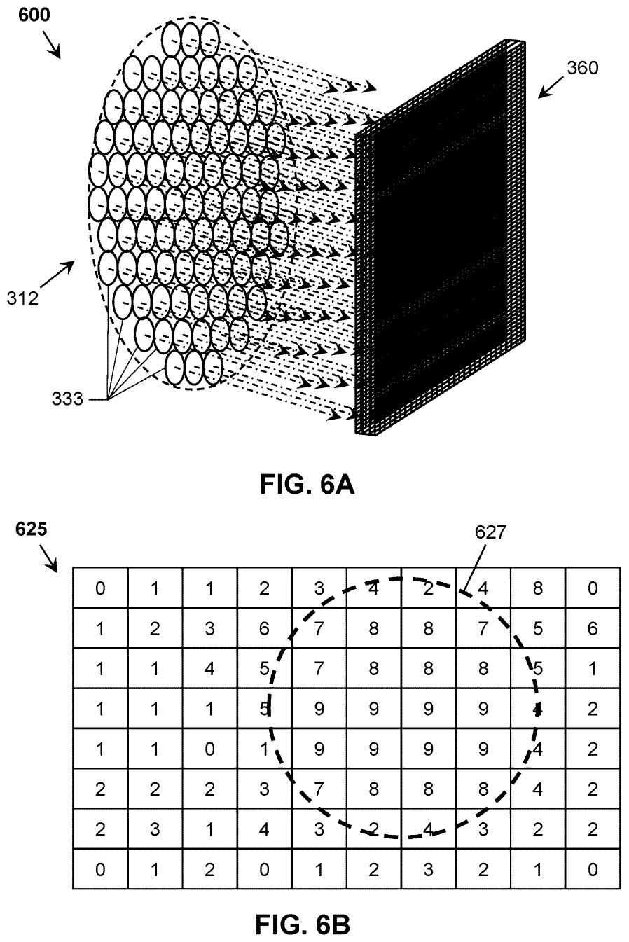

FIG. 6A illustrates the optical sensor 360 receiving electromagnetic radiation traveling through the plurality of optical fibers 306, in accordance with an embodiment 600 of the present invention. FIG. 6A illustrates the plurality of respective second optical endpoints 333, of the plurality optical fibers 306, bundled as the optical cord 312, impinging reflected radiation onto the optical sensor 360. The optical sensor 360 is embodied as a matrix of elements of a material that may be sensitive to a specific radiation characteristic such as the intensity, phase, polarization, wavelength shift, transit time, or several different combinations thereof. The same principles would also apply to the optical sensor 206.

Referring to FIG. 5, at Step 530, the processor 254 of the control module 204, or the processor 362 of the standalone controller 351 acting as a control module, determines radiation characteristics of the electromagnetic radiation received by the optical sensor 206 or 360, respectively. For example, depending on the intensity, phase, polarization, wavelength shift, transit time, etc., each element of the optical sensor 206 or 360 would show a correlated characteristic response that may be converted into a correlated electrical signal and transmitted to the processor 254 or the processor 362, respectively.

Furthermore, the processor 254 or the processor 362 on executing machine-readable instructions may access a location database stored in the storage device 260 or the memory unit 366, respectively. The location database is envisaged to include a plurality of first predefined location coordinate sets of the plurality of respective sensing locations 107 or 307, respectively, and a plurality of second predefined location coordinate sets of the plurality of respective stimulating locations 103 or 303, respectively, about a reference coordinate system. The reference coordinate system may be selected from a group consisting of a cartesian coordinate system, a polar coordinate system, a spherical coordinate system, and a cylindrical coordinate system.

The location database may also include a plurality of element location coordinate sets of the plurality of elements of the optical sensor 206 or 360, respectively. Moreover, a mapping may be provided between the plurality of first predefined location coordinate sets and the plurality of element location coordinate sets so that determined radiation characteristics from the plurality of elements may be assigned to the plurality of sensing locations 107 or 307, respectively.

FIG. 6B illustrates segment 625 of a matrix of radiation characteristics derived from the electromagnetic radiation received by the optical sensor 206 or 360, in accordance with an embodiment of the present invention. Each cell of the matrix is indicative of a sensing location. The radiation characteristics may correspond to intensity, phase shift, wavelength shift, transit time, polarization, etc. depending upon the type of optical sensor used and the type of analytical technique used.

Furthermore, in several embodiments of the invention, the processor 254 or the processor 362 determines at least one physiological parameter, such as local temperature, pressure, strain, visual abnormalities, etc. corresponding to each one of the plurality of first location coordinate sets indicative of the plurality of respective sensing locations 107 or 307, from the determined radiation characteristics. For example, the at least one physiological parameter may be determined using one or more analytical techniques deploying interferometers, Fiber Bragg gratings, confocal microscopy, Optical Coherence Tomography (OCT), fluorescence spectroscopy, Brillouin scattering, and Raman scattering. Moreover, the processor 254 or the processor 362 may utilize Machine Learning (ML) algorithms in combination with historical reference data for Artificial Intelligence (AI) based determination of the at least one physiological parameter.

In addition, the processor 254 or processor 362 stores the determined at least one physiological parameter of each one of the plurality of first location coordinate sets, in a storage device. The processor 254 may store the determined physiological parameter values in the storage device 260, while the processor 362 may temporarily store the physiological parameter values in the memory unit 366 and then transfer those values to an external storage device through the communication interface 368. However, in several embodiments of the invention, the therapeutic device 350 may also be provided with an internal storage device.

A region 627 of the segment 625 exhibits relatively high magnitudes of the determined radiation characteristics. The relatively high magnitudes may be indicative of a lesion, local tissue damage, redness, inflammation, cracks, or infections. Therefore, the processor 254 or the processor 362 would identify the region 627 as a region of target for providing the treatment. Referring to FIG. 5, at Step 540, the processor 254 or the processor 362, activates one or more of the plurality of stimulation elements 104 or 304 respectively. In that regard, the processor 254 or the processor 362 may again access the location database to identify the stimulation elements located in the region 627 and provide the stimulation best suited for the determined values of the at least one physiological parameter in the region 627. Such stimulation may include heating, cooling, irradiation, vibratory massage, ultrasonic treatment, electrical current application, or combinations thereof.

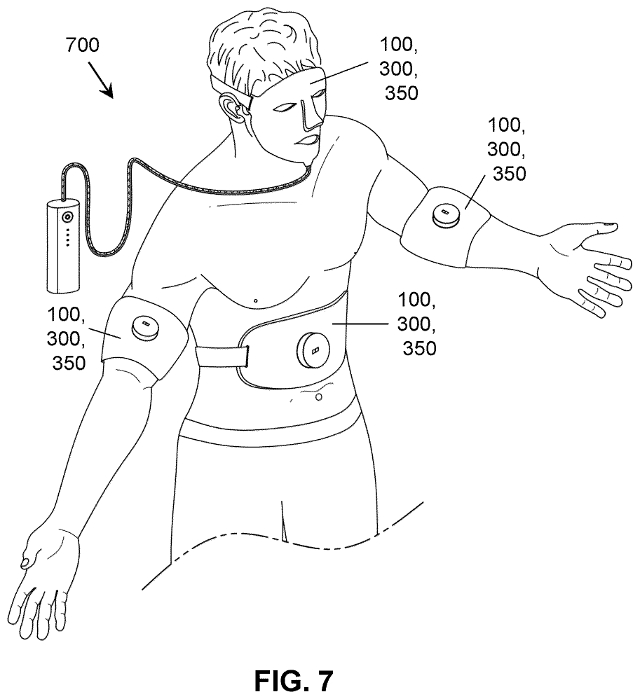

FIG. 7 illustrates a user 700 wearing several different embodiments of the therapeutic device 100, 300, or 350 depicting several possible application areas of the present invention. The therapeutic device 100, 300, or 350 may be embodied as a face mask, an armband, or a waistbelt.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapeutic device, comprising:
an inner layer comprising a plurality of sensing locations and a plurality of stimulating locations constructed therewithin, the inner layer configured to be in contact with skin of a user;
a plurality of stimulation elements located within the plurality of stimulating locations;
a plurality of optical fibers comprising a plurality of respective first optical endpoints and a plurality of respective second optical endpoints, the plurality of first optical endpoints located within the plurality of sensing locations;
an optical sensor configured to receive electromagnetic radiation traveling from the plurality of first optical endpoints to the plurality of second optical endpoints; and
a control module configured to determine radiation characteristics of the electromagnetic radiation received by the optical sensor and activate one or more of the plurality of stimulation elements in correlation with the determined radiation characteristics.

2. The therapeutic device as claimed in claim 1, further comprising a light source configured to provide incident radiation to the plurality of optical fibers through the plurality of second optical endpoints, wherein the electromagnetic radiation received by the optical sensor is reflected radiation from the plurality of first optical endpoints.

3. The therapeutic device as claimed in claim 1, wherein one or more of the plurality of sensing locations at least partially overlap with one or more of the plurality of stimulating locations.

4. The therapeutic device as claimed in claim 1, wherein the therapeutic device is embodied as one or more of a face mask, a waistbelt, and an armband.

5. The therapeutic device as claimed in claim 1, wherein the therapeutic device is made from one or more flexible materials.

6. The therapeutic device as claimed in claim 1, wherein the plurality of first optical endpoints are distributed amongst the plurality of sensing locations and the plurality of second optical endpoints are bundled in form of an optical cord.

7. The therapeutic device as claimed in claim 1, wherein one or more of the plurality of optical fibers comprises microcavity structures for temperature sensing.

8. The therapeutic device as claimed in claim 1, wherein the radiation characteristics are selected from a group consisting of intensity, phase, polarization, wavelength, transit time, and combinations thereof.

9. The therapeutic device as claimed in claim 1, wherein the plurality of stimulation elements is selected from a group consisting of irradiation sources, heating elements, cooling elements, vibration elements, ultrasonic wave generators, electrodes, and combinations thereof.

10. The therapeutic device as claimed in claim 9, wherein the irradiation sources are selected from a group consisting of Light Emitting Diodes (LEDs), and lasers.

11. The therapeutic device as claimed in claim 10, wherein the irradiation sources are configured to emit electromagnetic radiation in a range of wavelengths varying between 300 nm and 1200 nm.

12. The therapeutic device as claimed in claim 1, wherein one or more of the plurality of first optical endpoints comprise a parameter-sensitive coating thereupon, the parameter-sensitive coating being sensitive to one or more physiological parameters of the user.

13. The therapeutic device as claimed in claim 12, wherein the parameter-sensitive coating comprises a thermographic phosphor.

14. The therapeutic device as claimed in claim 1, wherein the control module is further configured to access a location database, the location database comprising a plurality of first predefined location coordinate sets of the plurality of respective sensing locations and a plurality of second predefined location coordinate sets of the plurality of respective stimulating locations, about a reference coordinates system.

15. The therapeutic device as claimed in claim 14, wherein the control module is further configured to:
determine at least one physiological parameter at each one of the plurality of first location coordinate sets indicative of the plurality of respective sensing locations, from the determined radiation characteristics; and
store the determined at least one physiological parameter of each one of the plurality of first location coordinate sets, in a storage device.

16. The therapeutic device as claimed in claim 15, wherein the at least one physiological parameter is selected from a group consisting of temperature, pressure, strain, and skin abnormalities.

17. The therapeutic device as claimed in claim 15, wherein the control module is configured to determine the at least one physiological parameter by utilizing interferometers, Fiber Bragg gratings, confocal microscopy, Optical Coherence Tomography (OCT), fluorescence spectroscopy, Brillouin scattering, and Raman scattering.

18. The therapeutic device as claimed in claim 15, wherein the control module is configured to utilize Machine Learning (ML) algorithms in combination with historical reference data for Artificial Intelligence (AI) based determination of the at least one physiological parameter.

19. A therapeutic device, comprising:
an inner layer comprising a plurality of sensing locations and a plurality of stimulating locations constructed therewithin, the inner layer configured to be in contact with skin of a user;
a plurality of stimulation elements located within the plurality of stimulating locations;
a plurality of optical fibers comprising a plurality of respective first optical endpoints and a plurality of respective second optical endpoints, the plurality of first optical endpoints located within the plurality of sensing locations;
an optical sensor configured to receive electromagnetic radiation traveling from the plurality of first optical endpoints to the plurality of second optical endpoints;
a light source configured to provide incident radiation to the plurality of optical fibers through the plurality of second optical endpoints, wherein the electromagnetic radiation received by the optical sensor is reflected radiation from the plurality of first optical endpoints; and
a control module configured to determine radiation characteristics of the electromagnetic radiation received by the optical sensor and activate one or more of the plurality of stimulation elements in correlation with the determined radiation characteristics.

20. A method of providing therapy, the method comprising:
providing a therapeutic device, the therapeutic device comprising:
an inner layer comprising a plurality of sensing locations and a plurality of stimulating locations constructed therewithin, the inner layer configured to be in contact with skin of a user,
a plurality of stimulation elements located within the plurality of stimulating locations,
a plurality of optical fibers comprising a plurality of respective first optical endpoints and a plurality of respective second optical endpoints, the plurality of first optical endpoints located within the plurality of sensing locations, an optical sensor configured to receive electromagnetic radiation traveling from the plurality of first optical endpoints to the plurality of second optical endpoints, and
a control module configured to determine radiation characteristics of the electromagnetic radiation received by the optical sensor and activate one or more of the plurality of stimulation elements in correlation with the determined radiation characteristics;
receiving, by the optical sensor, the electromagnetic radiation traveling from the plurality of first optical endpoints to the plurality of second optical endpoints;
determining, by the control module, the radiation characteristics of the electromagnetic radiation received by the optical sensor; and
activating, by the control module, the one or more of the plurality of stimulation elements in correlation with the determined radiation characteristics.
